(19) United States
(12) Patent Application Publication
(10) Pub. No.: US 2024/0366855 A1
(43) Pub. Date: Nov. 7, 2024

(54) ROLLING SURGICAL DRAIN WITH ANGLED DISTAL TIP

(71) Applicant: Early Bird Medical, Inc., Malvern, PA (US)

(72) Inventors: Robert KIEFER, Malvern, PA (US); E. Skott GREENHALGH, Gladwyne, PA (US)

(21) Appl. No.: 18/652,807

(22) Filed: May 1, 2024

Related U.S. Application Data
(60) Provisional application No. 63/499,475, filed on May 1, 2023.

(51) Int. Cl. A61M 1/00

(52) U.S. Cl. CPC A61M 1/915; A61M 1/916

(57) ABSTRACT

Surgical drains including a tubular mesh that is configured to distally expand from and proximally retract into a distal end of a tube or catheter. The tubular mesh may be expanded into a soft tissue body region where suction may be applied to drain fluid and other bodily material from the soft tissue body region. The tubular mesh is configured to invert along an inversion region of the tubular mesh as it is extended from and withdrawn into the tube or catheter. The distal end of the tube or catheter has a shape that promotes smooth exit of the tubular mesh out of the tube or catheter and/or entry of the tubular mesh into the tube or catheter.

### CLAIM OF PRIORITY

This patent application claims priority to U.S. Provisional Patent Application No. 63/499,475, titled “ROLLING SURGICAL DRAIN WITH ANGLED DISTAL TIP,” filed on May 1, 2023, and is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Surgical drains are used to remove fluid (blood, pus, etc.) and/or gas from a wound or body cavity. This broadly includes nasogastric tubes, urinary catheters, vascular access ports, ventriculoperitoneal shunts, and negative pressure surgical drains. In general, surgical drains can help the healing process by removing inflammatory mediators, bacteria, foreign material, and necrotic tissue. Drains can relieve pressure that can impair perfusion or cause pain, thereby decreasing morbidity and reducing inflammation. They enable monitoring for potential complications by allowing easy sampling of fluid during healing, and they can be used to address complications associated with dead space.

Negative pressure surgical drains, which are active surgical drains that use intermittent or continuous negative pressure to pull fluid and/or gas from a wound site or body cavity, are believed to provide advantages not realized with other types of surgical drains. Typically, passive drains are open systems and active drains are closed systems because they rely on negative pressure that is created by the drain.

Negative pressure drains may be particularly helpful in treating postpartum uterine bleeding. Postpartum uterine bleeding can occur when the uterine muscles are unable to achieve adequate contraction after delivery to cut off the blood flow that formerly circulated in the utero-placental space. The condition for this lack of contraction is called atony (lack of tone). The uterine muscles typically cut off the blood flow by contraction of the muscles to effectively pinch the arterial vessels that run through the tissue. In some cases, atony can result in arterial vessels that continue to bleed into the uterus (i.e., postpartum uterine bleeding). Postpartum hemorrhage, or excessive uterine blood loss after birth, is the leading cause of maternal death in the world. Inability to control postpartum bleeding can require a woman to receive multiple blood transfusions, and in severe cases, a full hysterectomy. Accordingly, it is desirable to control such postpartum bleeding. However, current medical devices and surgical procedures have proven inadequate in reducing postpartum hemorrhage or the amount of blood lost, and/or are extremely invasive.

One of the problems associated with negative pressure drains is that it can be difficult for negative pressure drains to provide uniform negative pressure within tissue cavities (both natural and those formed due to trauma), as soft tissue may collapse onto itself around the location(s) where pressure is applied, sealing off other regions from the pressure source. In addition, it may be difficult to remove the drain from tissue, particularly damaged and healing tissue, without causing further damage and disrupting nascent healing.

What is needed are negative pressure drains that can generate and sustain uniform regions of negative pressure within soft tissue, including, but not limited to the uterus, wounds and body cavities, without disrupting the apposition of tissue within the soft tissue and associated healing. In addition, it would be beneficial to have a negative pressure drain that is reliable and easy to use to minimize complications and procedure time.

### SUMMARY OF THE DISCLOSURE

The surgical drains and methods described herein provide negative pressure drains that can generate and sustain uniform regions of negative pressure within soft tissue. These apparatuses (devices, systems, drains, etc.) include a compliant mesh material that is disposed within a first elongate member (e.g., tube or catheter). During use, the mesh may be extended from the first elongate member and positioned within a wound or body cavity (e.g., uterus) where suction may be applied via the first elongate member to drain fluid (e.g., blood, lymph, pus, etc.) from the wound or body cavity. The first elongate member has a shaped distal end that facilitates extension of the mesh from the tube and/or retraction of the mesh into the first elongate member. The shaped distal end may prevent the mesh from jamming as it is extended from the first elongate member and/or retracted into the first elongate member, making the device easier to operate a traumatically within a body tissue, minimizing complications and procedure time.

In some examples, the surgical drains may include a second elongate member (e.g., inner shaft, rod, tube or catheter) that is configured to translate within the first elongate member and to control movement of the mesh in relation to the first elongate member. For example, the mesh may be in the form of tube in which a first end of the mesh is coupled to the first elongate member and a second end of the mesh is coupled to the second elongate member. Pushing the second elongate member may cause the mesh to extend distally past the distal end of the first elongate member so that the mesh may extend into the soft body tissue. The mesh may radially expand as it exits the first elongate member. Pulling the inner shaft may cause the mesh to invert and retract proximally past the distal end of the first elongate member and into a lumen of the first elongate member. The mesh may be radially compressed as it is retracted back into the first elongate member. The distal end of the first elongate member may be shaped to axially distribute stress that is placed on the mesh, thereby preventing bunching up of the mesh as it is extended from and/or retracted into the lumen of the first elongate member.

The shaped distal end of the first elongate member may be characterized as having any of a number of shapes. In some examples, the distal end includes one or more distally protruding portions (also referred to as protruding features or protrusions) that is/are configured to engage with the mesh as it extends from and/or retracts into the first elongate member. When the mesh transitions between the extended and retracted states, the mesh folds onto itself at an inversion region of the mesh. The distally protruding portion at the distal end of the tube may engage with the inversion region of the mesh to axially distribute a compressive force along the inversion region of the mesh. This can prevent the mesh from binding and jamming up as it unfolds from the first elongate member and/or folds into the first elongate member.

In some examples, the shaped distal end of the first elongate member has a tapered shape that defines the distally protruding portion. The tapered shape may be characterized as being at a non-perpendicular angle with respect to a longitudinal axis of the first elongate member. In some examples, the non-perpendicular angle may range anywhere from 1 degree to 89 degrees with respect to a longitudinal axis of the first elongate member. In some examples, the non-perpendicular angle ranges from about 30 degrees to about 45 degrees with respect to the longitudinal axis of the first elongate member.

The apparatus may be configured to apply a negative pressure (suction) through the first elongate member and the mesh. The mesh may be compliant and may distribute the negative pressure within the soft tissue region being treated. The mesh may be configured to take on a folded shape so that the mesh includes multiple layers through which the suction may be applied to provide multiple flow paths along the length of the mesh, e.g., into and between the porous layers of the mesh. When positioned within a soft tissue region (e.g., body cavity) with an applied negative pressure, the mesh may conform to the soft body tissue as it is drawn together while still maintaining a shape to allow fluid to flow through pores of the mesh, along the length of the mesh (e.g., between the layers) and to drain fluid from the soft tissue region. The apparatus may include one or more integrated or separate seals (e.g., plugs) that may seal off the soft tissue region so that the negative pressure may be sustained.

According to one example, a surgical drain system includes: a first elongate member having a distal end defining a distal opening that provides access to a lumen of the first elongate member, wherein the distal end includes a distally protruding portion; a second elongate member that is slidably disposed in the lumen; and a tubular mesh having a first end coupled to the first elongate member and a second end coupled to the second elongate member, wherein the mesh is configured to transition between an expanded configuration, in which the mesh is expanded distally past the distal end of the first elongate member, and a retracted configuration, in which the mesh is inverted and withdrawn within the lumen of the first elongate member, wherein the mesh is configured to invert at an inversion region as the mesh is extended from or retracted into the distal opening of the first elongate member, and wherein the distally protruding portion of the first elongate member is arranged to axially distribute stress along the inversion region of the mesh as the mesh is extended from or retracted into the distal opening, thereby reducing binding of the mesh.

The distal end of the first elongate member may have a tapered shape that defines the distally protruding portion. The tapered shape may have an angle ranging between 10 degrees and 45 degrees with respect to a longitudinal axis of the first elongate member. The tapered shape may have an angle ranging between 30 degrees and 45 degrees with respect to a longitudinal axis of the first elongate member. The tapered shape may have an angle ranging between 1 degree and 89 degrees with respect to a longitudinal axis of the first elongate member. The distally protruding portion of the first elongate member may be arranged to asymmetrically distribute the stress along an inversion plane of the inversion region of the mesh. The distally protruding portion of the first elongate member may be arranged to provide an axially elongated distal opening of the first elongate member. The distal end of the first elongate member may include multiple distally protruding portions arranged to asymmetrically distribute stress along the inversion region of the mesh as the mesh is extended from or retracted into the distal opening. The surgical drain system may further include a backstop configured to limit an extent to which the second elongate member is allowed to be pulled proximally. An outer surface of the first elongate member may include a plug that is configured to compress and expand, wherein when expanded, the plug is shaped to create a seal with surrounding soft tissue. The first end of the mesh may be coupled to a distal end region of the first elongate member, wherein the second end of the mesh is coupled to a distal end region of the second elongate member. The mesh may be coupled to an outer surface of the distal end region of the first elongate member. The mesh may be configured to transition from the expanded configuration to the retracted configuration upon application of a pulling force on the second elongate member. The mesh may be configured to transition from the retracted configuration to the expanded configuration upon application of a pushing force on the second elongate member. The first elongate member may be a tube. The second elongate member may be a tube or a rod. The lumen of the first elongate member may be configured to act as a vacuum channel configured to create a suction for removing fluid from a soft tissue cavity when the mesh is positioned within the soft tissue cavity. The vacuum channel may extend between the distal end of the first elongate member and one or more proximal vacuum ports of the first elongate member. The vacuum channel may be within a space between an outer surface of the second elongate member and an inner surface of the first elongate member. A lumen of the second elongate member may be configured to act as a vacuum channel configured to create a suction for removing fluid from a soft tissue cavity when the mesh is positioned within the soft tissue cavity.

According to another example, a method of draining a body region includes: positioning a surgical drain apparatus into a soft tissue channel that leads to the body region, wherein the surgical drain apparatus includes a first elongate member having a distal end defining a distal opening that provides access to a lumen of the first elongate member, wherein the distal end includes a distally protruding portion, and wherein a tubular mesh is housed within the lumen of the first elongate member; distally extending the tubular mesh from the lumen of the first elongate member and into the body region, wherein distally extending the tubular mesh causes the tubular mesh to transition from an inverted configuration to a non-inverted configuration, wherein the tubular mesh inverts at an inversion region as the mesh is extended from distal opening of the first elongate member, and wherein the distally protruding portion of the first elongate member axially distributes stress along the inversion region of the mesh as the mesh is extended from the distal opening, thereby preventing binding of the mesh; and applying suction through the lumen so that a plurality of flow paths are created through the mesh, wherein the suction drains material proximally from the body region through the mesh and the first elongate member.

The method may further include, after applying sufficient suction to the body region, retracting the tubular mesh into the first elongate member, wherein the distally protruding portion of the first elongate member asymmetrically distributes the stress along the inversion region of the mesh as the mesh is retracted into the distal opening, thereby preventing binding of the mesh. The method may further include, prior to applying the suction, creating a scal around the first elongate member with the soft tissue channel to maintain a vacuum within the body region. The surgical drain apparatus may further include a second elongate member slidably disposed within the lumen of the first elongate member and coupled to the tubular mesh, wherein distally extending the tubular mesh from the lumen of the first elongate member comprises pushing the second elongate member distally. The distal end of the first elongate member may have a tapered shape that defines the distally protruding portion. The tapered shape may have an angle ranging between 10 degrees and 45 degrees with respect to a longitudinal axis of the first elongate member. The tapered shape may have an angle ranging between 30 degrees and 45 degrees with respect to a longitudinal axis of the first elongate member. The tapered shape may have an angle ranging between 1 degree and 89 degrees with respect to a longitudinal axis of the first elongate member. The may have distally protruding portion of the first elongate member may asymmetrically distribute the stress along an inversion plane of the inversion region of the tubular mesh. The distally protruding portion of the first elongate member may axially elongate the distal opening of the first elongate member. The distal end of the first elongate member may include multiple distally protruding portions that asymmetrically distribute the stress along the inversion region of the mesh as the mesh is extended from the distal opening.

These and other examples are described in detail herein.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates a side view of an example surgical drain with an invertible tubular mesh.

FIGS. 2A and 2B illustrate close-up side views of an example first elongate member of a surgical drain, where the first elongate member has a perpendicular shaped distal end.

FIGS. 3A and 3B illustrate close-up side views of an example first elongate member of a surgical drain, where the first elongate member has a shaped distal end that prevents binding of a mesh that is extended out of and/or retracted into the first elongate member.

FIGS. 4A-4C illustrate section views of an example surgical drain with a first elongate member having a shaped distal end: FIG. 4A shows the mesh in a fully extended configuration; FIG. 4B shows the mesh in an extended double-walled configuration; and FIG. 4C shows the mesh in a retracted/withdrawn configuration.

FIGS. 5A and 5B illustrate side views of another example surgical drain with a first elongate member having a shaped distal end for preventing mesh binding: FIG. 5A shows the mesh in a retracted/withdrawn configuration; and FIG. 5B shows the mesh in an extended double-walled configuration.

FIGS. 6A and 6B illustrate side views of another example surgical drain with a first elongate member having a perpendicular shaped distal end: FIG. 6A shows the mesh in a retracted/withdrawn configuration; and FIG. 6B shows the mesh in an extended double-walled configuration.

FIGS. 7A and 7B illustrate side views of another example surgical drain with a first elongate member having a shaped distal end for preventing mesh binding: FIG. 7A shows the mesh in a retracted/withdrawn configuration; and FIG. 7B shows the mesh in an extended double-walled configuration.

FIGS. 8A and 8B illustrate side views of another example surgical drain with a first elongate member having a perpendicular shaped distal end: FIG. 8A shows the mesh in a retracted/withdrawn configuration; and FIG. 8B shows the mesh in an extended double-walled configuration.

FIGS. 9A and 9B illustrate side and perspective views, respectively, of an example shaped distal end of a first elongate member, where the shaped distal end has protruding portion and a perpendicular portion.

FIGS. 10A and 10B illustrate side and perspective views, respectively, of an example shaped distal end of a first elongate member, where the shaped distal end has two symmetrically arranged protruding portions.

FIGS. 11A and 11B illustrate side and perspective views, respectively, of an example shaped distal end of a first elongate member, where the shaped distal end has a flat protruding portion.

FIGS. 12A and 12B illustrate side and perspective views, respectively, of an example shaped distal end of a first elongate member, where the shaped distal end has two asymmetrically arranged protruding portions.

FIGS. 13A and 13B illustrate side and perspective views, respectively, of an example shaped distal end of a first elongate member, where the shaped distal end has a cradled shaped protruding portion.

FIGS. 14A and 14B illustrate side and perspective views, respectively, of an example shaped distal end of a first elongate member, where the shaped distal end has two symmetrically arranged protruding portions.

FIGS. 15A and 15B illustrate side and perspective views, respectively, of an example shaped distal end of a first elongate member, where the shaped distal end has two rounded protruding portions.

FIGS. 16A and 16B illustrate side and perspective views, respectively, of an example shaped distal end of a first elongate member, where the shaped distal end has a rounded protruding portion.

FIGS. 17A-17D illustrates an example use of an example surgical drain in a soft tissue region of a body: FIG. 17A shows a first cross sectional view of the soft tissue region; FIG. 17B shows a second cross sectional view of the soft tissue region; FIG. 17C shows the surgical drain after a mesh is inserted within the body region and as a negative pressure is applied; and FIG. 17D shows the mesh in the body region after the body region is at least partially contracted.

FIGS. 18A and 18B illustrate an example surgical drain having a plug for scaling with soft tissue: FIG. 18A shows a cross section view of a body region that includes a relatively large channel; and FIG. 18B shows a surgical drain inserted within the body region, where the surgical drain includes a plug to seal the relatively large channel.

FIGS. 19A and 19B illustrate an example plug assembly for a surgical drain: FIG. 19A shows a transparent side view of the plug in a radially expanded state; and FIG. 19B shows a transparent side view of the plug in a radially compressed state.

FIG. 20 is a flowchart illustrating an example method of treating a body region using a surgical drain.

### DETAILED DESCRIPTION

Described herein are methods and apparatuses for draining a body region of a subject in order to remove fluid and/or other material from the body region. In some cases, the apparatus may additionally or alternatively be used to contract surrounding tissues of the body region. This treatment may prevent or reduce bleeding and/or may otherwise enhance healing. These apparatuses and methods, including methods of using them may, may be particularly useful for forming regions of uniform negative pressure within soft tissue, and sustaining the negative pressure while atraumatically deploying and/or removing a portion of the apparatus (e.g., mesh) from within the soft tissue.

For example, described herein are apparatuses, including surgical drain systems, that include an invertible porous mesh that may be expanded within the body region being treated, and may be used to distribute the negative pressure (e.g., suction) applied by the apparatus within the body region being treated. For example, the negative pressure may be applied to the body region (e.g., chamber, pocket, sac, etc.) via the pores of the mesh. The apparatuses may also include a seal or closure that may allow the treated body region to retain the negative pressure so that adequate suction can be formed to remove fluid and/or other materials away from the body region. The apparatuses may be configured so that once the negative pressure is applied (and maintained) the mesh may be gently removed by inverting the mesh over itself and drawing it into the apparatus. The peeling removal force will generally be lower (by one or more orders of magnitude) than a drag force needed for other surgical drains.

Further, the mesh may be formed of a porous textile, fabric, or sheath material. The mesh typically communicates with an inner drain (e.g., a vacuum channel that leads to one or more vacuum ports) so that negative pressure may be applied to the mesh when positioned with the body region. This may allow the mesh to distribute negative pressure to a greater area and/or to create a larger surface area for fluid control. The porosity (e.g., the space between filaments in variations in which the mesh is formed of knitted, woven or braided fibers) may be controllable. Any of these meshes may be self-expanding (e.g., formed of a material such as Nitinol, Nitinol mixed with one or more polymers, etc.).

Any of the apparatuses described herein may have axial flexibility, so that it can be bent around structures or non-uniform volumes. In some variations the apparatus may be introduced into a body orifice through a native or natural channel, such as for treating a uterus by passing through the vaginal canal.

In some examples, the mesh is in the form of a tube. For example, a tubular mesh may include walls formed around a central axis to define a generally tubular shape. A first end of the tubular mesh may be coupled to the first elongate member (e.g., tube or catheter) and a second end of the tubular mesh may be coupled to a second elongate member (e.g., shaft, rod, or second tube or catheter). The tubular mesh may be configured to transition between an expanded configuration, in which the mesh is expanded distally past the distal end of the first elongate member, and a retracted configuration, in which the mesh is withdrawn within the lumen of the first elongate member. The tubular mesh may be configured to invert when retracted into the first elongate member.

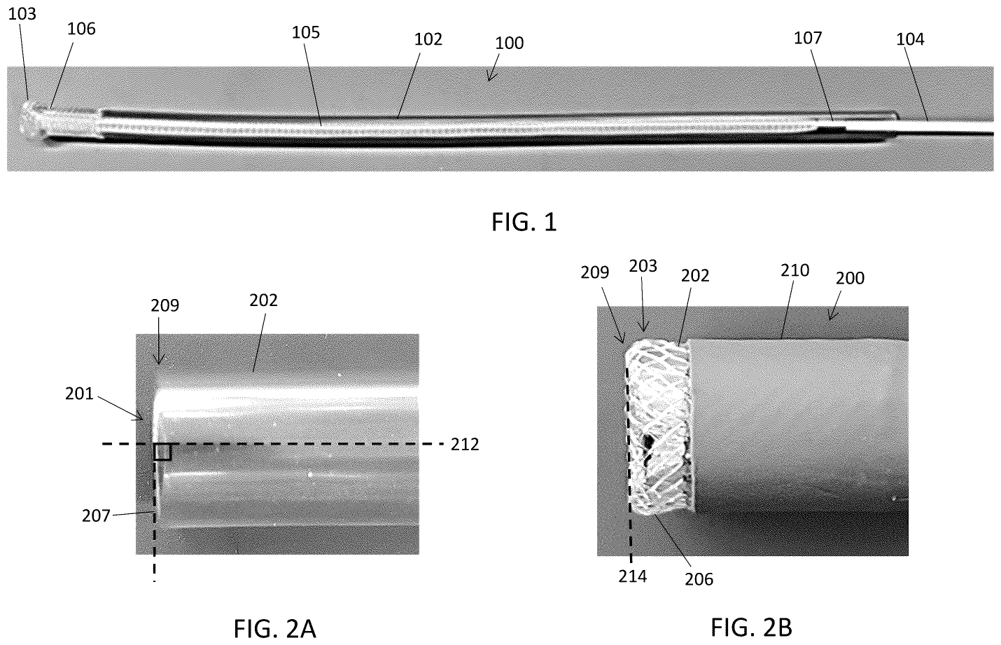

FIG. 1 shows an example surgical drain 100 having an invertible tubular mesh 106. The surgical drain 100 includes a first elongate member 102 (e.g., tube or catheter) that includes an inner lumen. A second elongate member 104 (e.g., shaft, rod, or second tube or catheter) is slidably disposed within the lumen of the first elongate member 102. The mesh 106 has a first end that is coupled to the first elongate member 102, and a second end that is coupled to the second elongate member 104. For example, the mesh 106 may be glued, melted, welded, molded and/or attached via one or more fasteners or bands to the first elongate member 102 and/or the second elongate member 104. In the example shown, the first end of the mesh 106 is coupled to an outer surface of a distal end region of the first elongate member 102. Also in the example shown, the second end of the mesh 106 is coupled to a distal end region of the second elongate member 104 via a fastener 107.

The surgical drain 100 (and the mesh 106) is configured to transition between an expanded configuration, in which the mesh 106 is expanded distally past the distal end of the first elongate member 102, and a retracted configuration, in which the mesh 106 is inverted and withdrawn within the lumen of the first elongate member 102. FIG. 1 shows the surgical drain 100 (and the mesh 106) in a retracted configuration in which at least a portion 105 of the mesh 106 is withdrawn and in an inverted state within the lumen of the first elongate member 102.

During use, the surgical drain 100 may be positioned near or within a body region (e.g., wound, body cavity, body channel) while the mesh 106 is in a retracted configuration. Once positioned, the mesh 106 may be extended distally into the body region. The mesh 106 may be configured to expand radially as it is released from the first elongate member 102. This may allow the mesh 106 to expand into and occupy more of the volume of the body region (e.g., wound, body cavity, body channel), which may prevent local regions of higher negative pressure to develop and thereby providing more uniform draining. The surgical drain 100 may include one or more seals to create a seal between the body region with the lumen of the first elongate member 102. Once a seal is established, suction may be applied to the sealed volume to cause fluid and other bodily material to drain from the body region. After draining is complete, the mesh 106 may be retracted back into the first elongate member 102 and the surgical drain 100 may be removed from the subject.

Distal and proximal movement of the mesh 106 relative to the distal end of the first elongate member 102 may be controlled by relative translation between the first elongate member 102 and the second elongate member 104. For example, from the retracted configuration shown in FIG. 1, the second elongate member 104 may be pushed distally to cause the mesh 106 to move distally out of the first elongate member 102. As the mesh 106 is distally extended, it continuously inverts at an inversion region 103 of the mesh 106 until the pushing force is stopped. This continuous inversion of the mesh 106 may be referred to as “rolling.” The inversion causes at least a portion of the inside of the mesh 106 to face outward and the outside of the mesh 106 to face inward. That is, the mesh 106 may transition from a first tubular state (e.g., inside out state) within the first elongate member 102 to a second tubular state (e.g., outside out state) when extended out of the first elongate member 102. From the expanded configuration, the second elongate member 104 may be pulled proximally to cause the mesh 106 to move proximally and invert back into the first elongate member 102.

The amount of extension of the mesh 106 outside of the first elongate member 102 may be controlled by translation of the second elongate member 104 by the user. In some cases, the surgical drain 100 may include one or more stops to control the extent to which the second elongate member 104 may translate with respect to the first elongate member 102. For example, the surgical drain 100 may include a back stop to limit the extent to which the second elongate member 104 may translate proximally, which may be useful in preventing a user from pulling the second elongate member 104 too far proximally. Alternatively or additionally, the surgical drain 100 may include a front stop to limit the extent to which the second elongate member 104 may translate distally, which may be useful in preventing a user from pushing the second elongate member 104 too far distally. In some cases, the surgical drain 100 may be configured to extend distally until all (or at least a majority) of the mesh 106 extends outside of the first elongate member 102 and the mesh 106 is fully (or at least mostly) not in the inverted state.

As described in detail later, the inversion region 103 may be at different axial locations along the mesh 106 as the mesh 106 is extended and withdrawn from the first elongate member 102. For example, the inversion region 103 may be positioned at or near the distal end of the first elongate member 102 when the mesh 106 is fully withdrawn (or almost fully withdrawn) into the first elongate member 102, and positioned more distally with respect to the distal end of the first elongate member 102 when the mesh 106 is more distally extended. In some cases, the mesh 106 may bunch up at the inversion region 103 when inversion region 103 is at or near the distal end of the first elongate member 102, causing the mesh 106 to jam.

FIGS. 2A and 2B show close-up views of a distal end 209 of a first elongate member 202 of an example surgical drain, illustrating bunching up a mesh 206. FIG. 2A shows the distal end 209 (also referred to as a distal edge or distal tip) of the first elongate member 202 (e.g., tube or catheter), which includes a distal opening 201 that provides access to the lumen of the first elongate member 202. As shown, the distal end 209 is perpendicular with respect to a longitudinal axis 212 of the first elongate member 202. More specifically, a plane 207 defined by the distal end 209 is perpendicular (at a 90 degree angle) with respect to the longitudinal axis 212 of the first elongate member 202.

FIG. 2B shows the mesh 206 having a first end coupled to the outside of the first elongate member 202 (e.g., by glue) and further secured by a compression band 210. In the example shown, the mesh is in a retracted configuration, in which an inversion region 203 of the mesh 206 folds along an inversion plane 214 over the distal end 209 of the first elongate member 202 and a remainder of the mesh 206 is disposed within the lumen of the first elongate member 202. In this configuration, the material of the mesh 206 along the inversion region 203 is radially compressed from being confined by the diameter of distal opening 201 of the first elongate member 202, thereby bunching up the mesh 206 in an axial direction and inducing a compressive stress on the inversion region 203 of the mesh 206. In some cases, this bunching up and stress may create a resistance (e.g., friction) that causes the mesh 206 to bind as it is retracted into the first elongate member 202 and/or as it is extended out of the first elongate member 202. This may occur, for example, when the mesh 106 is left in the retracted/collapsed configuration for a long period of time. A user may need to pull or push the second elongate member (e.g., 104 in FIG. 1) harder when the inversion region 203 is near or at the distal end 209 of the first elongate member 202. In some cases, the user may not be able to extend the mesh 206 and/or fully retract the mesh 206.

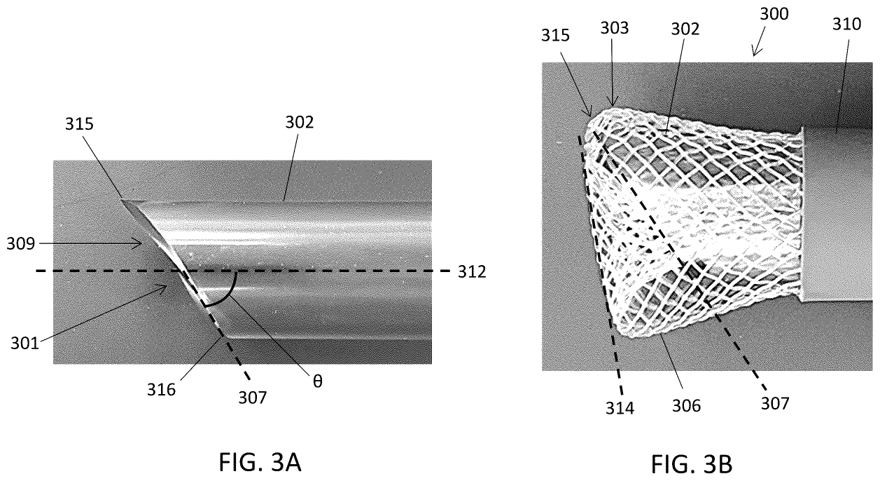

FIGS. 3A and 3B show a distal end 309 of a first elongate member 302 of another example surgical drain 300, illustrating one solution to the mesh binding problem. FIG. 3A shows the distal end 309 of a first elongate member 302 that includes a distally protruding portion 315 (also referred to as a protruding feature or protrusion) that protrudes distally more than, for example, a distally recessed portion 316 of the distal end 309. In this example, the distal end 309 is cut at a non-perpendicular with respect to a longitudinal axis 312 of the first elongate member 302. For example, a plane 307 defined by the distal end 309 of the first elongate member 302 is at a non-perpendicular angle θ with respect to the longitudinal axis 312 of the first elongate member 302. The distally protruding portion 315 effectively elongates a distal opening 301 of the first elongate member 302 in the axial direction (e.g., compared to the perpendicular distal end 209 in FIGS. 2A-2B).

FIG. 3B shows a mesh 306 having a first end coupled to the outside of the first elongate member 302 (e.g., by glue) and further secured by a compression band 310. An inversion region 303 of the mesh 306 folds over the distal end 309 of the first elongate member 302 so that a remainder of the mesh 306 is disposed within the lumen of the first elongate member 302. As the mesh 316 is pushed distally (e.g., by pushing the second elongate member) or pulled proximally (e.g., by pulling a second elongate member), it engages with the protruding portion 315 of the distal end 309 and creates more slack in the mesh 306 near the recessed portion 316. In this way, the stress may be concentrated at the portion of the mesh 306 folding over the protruding portion 315 of the first elongate member, thereby providing non-symmetrical propagation of stress during unrolling of the mesh 306. This asymmetric distribution of stress may allow the portion of the mesh 306 experiencing less stress (e.g., near the recessed portion 316 of the first elongate member 302) to enter the distal opening 301 more easily. Further, the axially elongated distal opening 301 may provide more room for the mesh 306 to invert as it enters or exits the distal end 309 of the first elongate member 302, thereby further reducing the chances of binding/jamming of the mesh 306.

The shape of the distal end 309 of the first elongate member 302 may also influence the shape of the inversion region 303 of the mesh 306. For example, an inversion plane 314 of the inversion region 303 may be at a non-perpendicular angled relative to the longitudinal axis 312 of the first elongate member 302. As described later, the inversion plane 314 may remain non-perpendicular with respect to the longitudinal axis 312 of the first elongate member 302 even as the mesh 306 is extended to an expanded configuration.

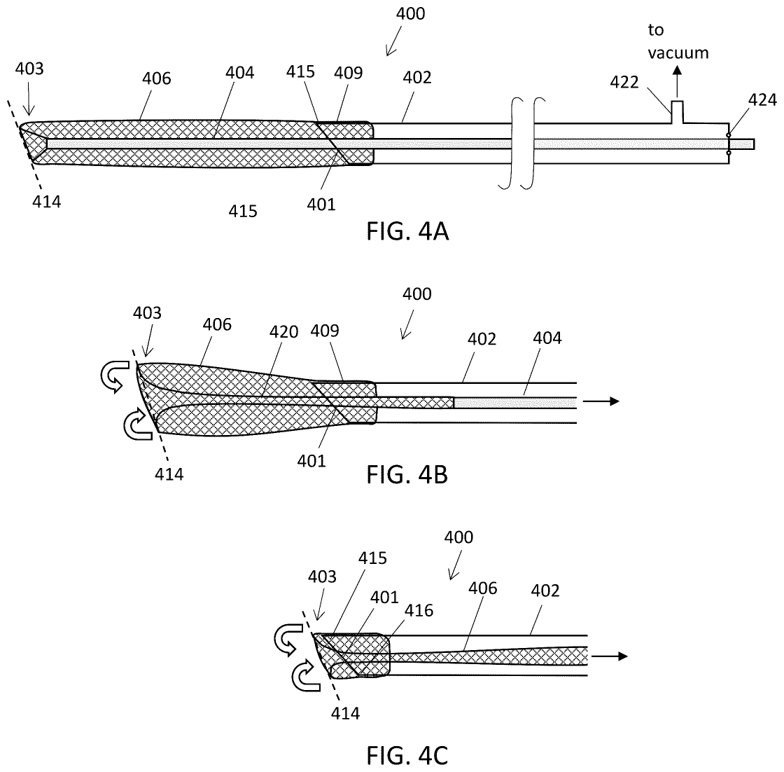

FIGS. 4A-4C show an example of a surgical drain 400 having a first elongate member 402 with a shaped distal end 409 to reduce binding of an invertible tubular mesh 406. A first end of the mesh 400 is coupled to a distal end region of a first elongate member 402, and a second end of the mesh 400 is coupled to a distal end region of a second elongate member 404. The lumen of the first elongate member 402 forms the vacuum channel extending from a proximal vacuum port 422 to one or more distal openings at the distal end of the first elongate member 406. The vacuum port 422 is configured to connect to a source of negative pressure (e.g., vacuum). In some examples the vacuum port 422 may be a mating connection (a scaling mating connection) to couple to tubing for connecting to the source of negative pressure. The lumen of the first elongate member 402 is sealed at the proximal end by one or more seals 424 (e.g., O-ring). In some cases the vacuum port 422 may include a lock (e.g., luer-type lock) for opening/closing (to allow on/off of the negative pressure, and/or to maintain or hold the pressure already applied). In this example, the vacuum port 422 is formed on the outer surface of the first elongate member 402 at the proximal end region. Alternatively the vacuum port 402 may extend from the first elongate member 402 via a tube or channel. In some examples, the vacuum source may be coupled to a lumen of the second elongate member 404 (which may be a tube) may provide a vacuum channel in addition to (or instead of) the vacuum channel of the first elongate member 402.

FIG. 4A shows the surgical drain 400 in an extended configuration where the second elongate member 404 has been pushed distally relative to the first elongate member 402 to almost fully extend the mesh 406 distally except for at an inversion region 403 at a distal end of the surgical drain 400. In this extended configuration, the mesh 406 may take on a tubular shape. The mesh 406 may radially expand when released from the first elongate member 402. The inversion region 403 may define an inversion plane 414 that is at a non-perpendicular angle with respect to the longitudinal axis of the first elongate member 402.

From the extended state in FIG. 4A, the second elongate member 404 may be pulled proximally to cause the mesh 406 to fold onto itself along the inversion region 403 as it is retracted within a distal opening 401 of the first elongate member 402, as shown in FIG. 4B. In the partially inverted and expanded state shown in FIG. 4B, the walls of the mesh 406 double back forming a double-walled tubular shape that defines a second lumen 420 formed by mesh 406. In some cases, the surgical drain 400 may be configured to apply suction such that fluid and/or body material flows into the second lumen 420 and out of the mesh 406 in the proximal direction. In the example shown in FIG. 4B, the second elongate member 404 is withdrawn proximally past the distal end 409 of the first elongate member 402, which may allow the portion of the mesh 406 that extends outside of the first elongate member 402 to have more lateral flexibility (e.g., compared to when the second elongate member 404 is distally extended past the distal end 409 of the first elongate member 402). This may allow the mesh 406 to bend laterally more easily during use, for example, as it contacts tissue walls within the body cavity. In some examples, a distal end of the second elongate member 404 is positioned near the distal end 409 of the first elongate member 402 to maximize a length of the mesh 406 in the double-walled tubular configuration.

FIG. 4C shows the surgical drain 400 as the second elongate member 404 is pulled further proximally such that the mesh 406 is almost fully inverted and withdrawn within the first elongate member 404, thereby mostly taking on a tubular shape that is inverted (compared to the non-inverted state in FIG. 4A). As the mesh 406 is pulled proximally (e.g., by pulling a second elongate member), the inversion region 403 of the mesh 406 engages with the protruding portion 415 of the distal end 409 of the first elongate member 402, thereby creating a localized region of greater stress on the mesh 406 at the protruding portion 415 of the first elongate member 402. This allows for lesser stress placed on other portions of the inversion region 403 of the mesh 406 along the inversion plane 414 (e.g., portions of the mesh 406 at a recessed portion 416 of the first elongate member 402). The portions of the inversion region 403 experiencing lesser stress may more easily enter the distal opening 401 of the first elongate member 402, thereby reducing the likelihood that the mesh 406 will bind along the inversion region 403. In this way, the protruding portion 415 is arranged to asymmetrically distribute stress along the inversion region 403 of the mesh 406 as it is retracted into the distal opening 401 of the first elongate member 402. The protruding portion 415 may similarly asymmetrically distribute stress along the inversion region 403 of the mesh 406 as it is extended from the distal opening 401 of the first elongate member 402 when transitioning from the retracted configuration (FIG. 4C) to an extended configuration (e.g., FIG. 4A or 4B). Further, the protruding portion 415 axially elongates the distal opening 401 of the first elongate member 402, thereby providing more room for the mesh 406 to invert and radially compress as it enters or exits the distal end 409 of the first elongate member 402. These factors may reduce the likelihood of binding/jamming of the mesh 406 as it enters or exits the distal end 409 of the first elongate member 402.

The surgical drain 400 may be configured to apply a negative pressure on the surgical drain 400 at any state of mesh 406 extension/inversion. For example, it may be beneficial to apply suction when the mesh 406 is almost fully extended distally (e.g., FIG. 4A) to access more distal regions of a body cavity. Alternatively or additionally, it may be beneficial to apply suction when the mesh 406 is in a double-walled state (e.g., FIG. 4B) where the mesh 406 may be more flexible due to the second elongate member 402 being withdrawn, thereby allowing the mesh 406 to conform easier to the geometry of a body cavity.

In some cases, the first elongate member 402 and/or the second elongate member 404 may include one or more stops and/or locks to limit their relative axial movement and/or lock their relative axial positions. For example, the mesh 406 may be stopped and/or locked in the double-walled tubular configuration, such as shown in FIG. 4B. Additionally or alternatively, the mesh 406 may be stopped and/or locked in a distally extended configuration where the second elongate member 404 is distally extended with respect to the first elongate member 402, such as shown in FIG. 4A, and/or in a withdrawn configuration where the mesh 406 is inverted and withdrawn into the lumen of the first elongate member 402, such as shown in FIG. 4C.

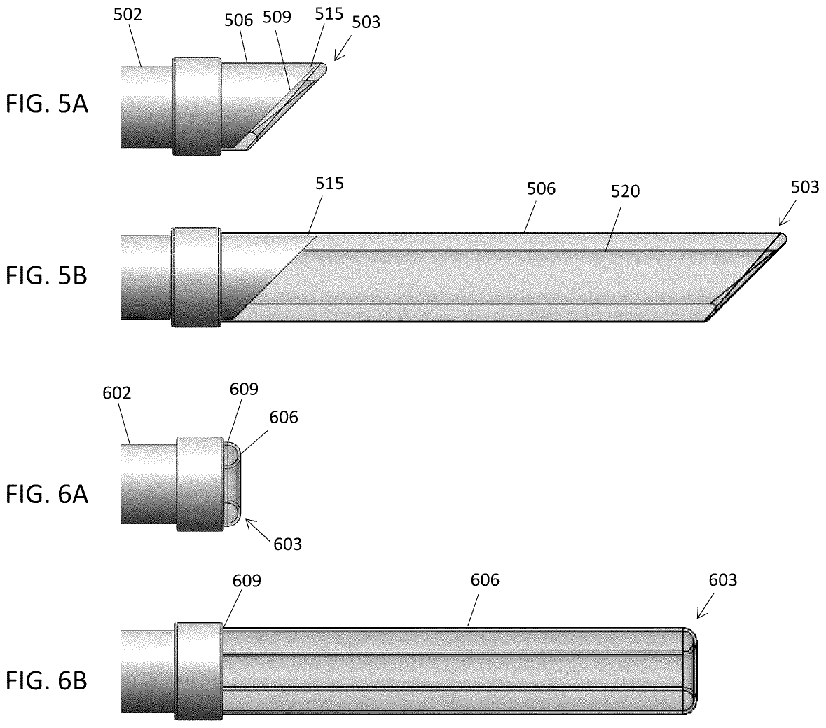

FIGS. 5A-5B and 6A-6B illustrate another comparison between a surgical drain with a first elongate member having distally protruding portion (FIGS. 5A-5B) and a surgical drain with a first elongate member without a distally protruding portion (FIGS. 6A-6B).

In FIGS. 5A and 5B, the surgical drain includes a first elongate member 502 having a tapered distal end 509 defining a distally protruding portion 515. FIG. 5A shows an invertible tubular mesh 506 in a retracted configuration such that an inversion region 503 of the mesh 506 engages with a least a portion of the distal end 509 of the first elongate member 502. As discussed herein, the distally protruding portion 515 can focus the compressive force placed mesh 506 as it is extended from or retracted into the first elongate member 502. This asymmetrically distribution of stress along the inversion region 503 of the mesh 506 can reduce binding of the mesh 506, for example, as the mesh 506 is extended to an expanded configuration as shown in FIG. 5B. Suction may be applied to the surgical drain in this expanded configuration to remove fluid and other bodily material from a body cavity via pores in the walls of the mesh 506. In addition, the mesh 506 takes on a double-walled tubular configuration (similar to FIG. 4B) that defines a second lumen 520 through which fluid and other bodily material may also be suctioned through. As shown, the inversion region 503 of the mesh 506 may take on a non-perpendicular shape in the retracted configuration of FIG. 5A and in the expanded configuration of FIG. 5B.

In FIGS. 6A and 6B, the surgical drain includes a first elongate member 602 having a perpendicular distal end 609 (i.e., having no distally protruding portion). FIG. 6A shows an invertible tubular mesh 606 in a retracted configuration such that an inversion region 603 of the mesh 606 is engaged with the perpendicular distal end 609 of the first elongate member 602. In contrast to the surgical drain of FIGS. 5A and 5B, the inversion region 603 of the mesh 606 is axially aligned at the distal end 609 of the first elongate member 602. This may create a compressive stress on the inversion region 603 of the mesh 606 sufficient cause the mesh 606 to bind/jam at as it is extended from and/or retracted into the first elongate member 602. As shown, the inversion region 603 of the mesh 606 may also have a perpendicular shape in the retracted configuration of FIG. 6A and in the expanded configuration of FIG. 6B.

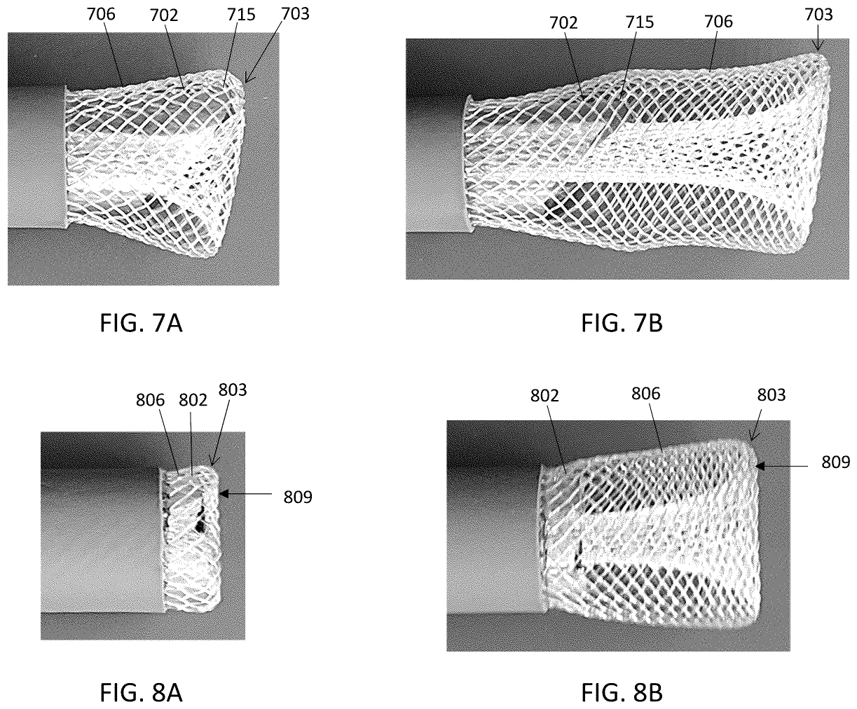

FIGS. 7A-7B and 8A-8B illustrate yet another comparison between a surgical drain with a first elongate member having distally protruding portion (FIGS. 7A-7B) and a surgical drain with a first elongate member without a distally protruding portion (FIGS. 8A-8B).

In FIGS. 7A and 7B, the surgical drain includes a first elongate member 702 having a tapered distal end defining a distally protruding portion 715. FIG. 7A shows an invertible tubular mesh 706 in a retracted configuration such that an inversion region 703 of the mesh 706 engages with a least the distally protruding portion 715 of the first elongate member 702. The distally protruding portion 715 can focus the compressive force placed mesh 706 as it is extended from or retracted into the first elongate member 702, which can reduce binding of the mesh 706. FIG. 7B shows the mesh 706 in an extended double-walled tubular configuration.

In FIGS. 8A and 8B, the surgical drain includes a first elongate member 802 having a perpendicular distal end 809 (i.e., having no distally protruding portion). FIG. 8A shows an invertible tubular mesh 806 in a retracted configuration such that an inversion region 803 of the mesh 806 is engaged with the perpendicular distal end 809 of the first elongate member 802. The inversion region 803 is axially aligned at the distal end 809 of the first elongate member 802, which may create a compressive stress on the mesh 806 that is sufficient cause the mesh 806 to bind/jam at as it is extended from and/or retracted into the first elongate member 802.

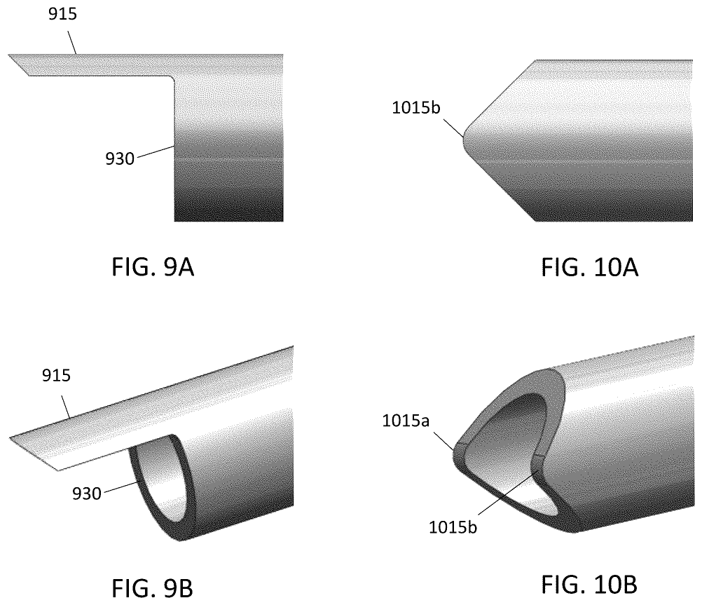

In any of the apparatuses described herein, the distal end of the first elongate member may include one or more protruding portions (e.g., 1, 2, 3, or more) having any of a number of shapes. FIGS. 9A-16B show example distal ends of first elongate members having protruding portions with different shapes. The first elongate members of the devices described herein may include any number of protruding portions having any of a number of shapes, and are not limited to the examples presented in FIGS. 9A-16B.

FIGS. 9A and 9B show an example distal end of a first elongate member having a protruding portion 915 and a perpendicular portion 930. The protruding portion 915 may asymmetrically distribute stress along an inversion region of the mesh by creating two points of stress concentration.

FIGS. 10A and 10B show an example distal end of a first elongate member having two protruding portions 1015a and 1015b, which may asymmetrically distribute stress along an inversion region of the mesh by creating two points of stress concentration. In this example, the protruding portions 1015a and 1015b are symmetrically arranged with respect to a longitudinal axis of the first elongate member.

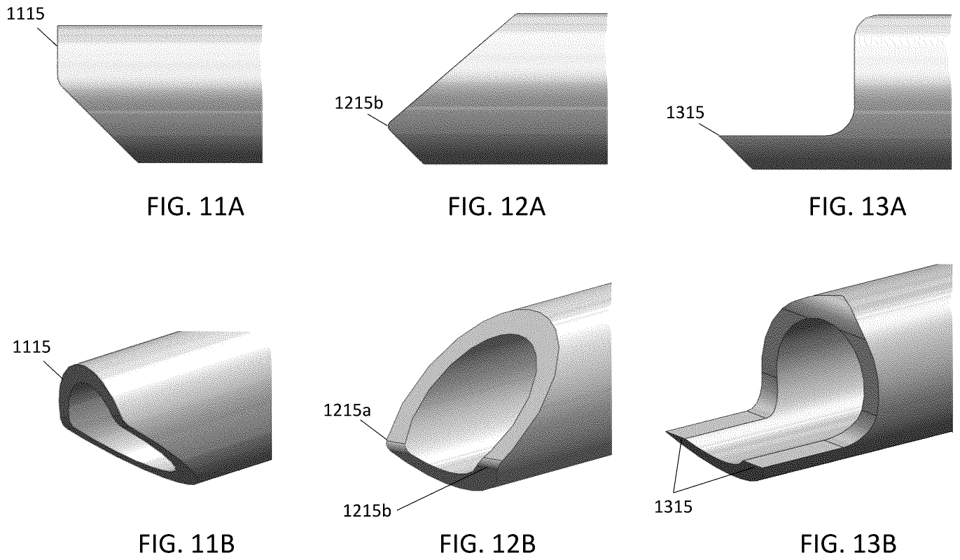

FIGS. 11A and 11B show an example distal end of a first elongate member having protruding portion 1115 with a flat (e.g., perpendicular) engagement surface.

FIGS. 12A and 12B show an example distal end of a first elongate member having two protruding portions 1215a and 1215b that are asymmetrically arranged with respect to a longitudinal axis of the first elongate member.

FIGS. 13A and 13B show an example distal end of a first elongate member having a protruding portion 1315 having a cradle-like shape.

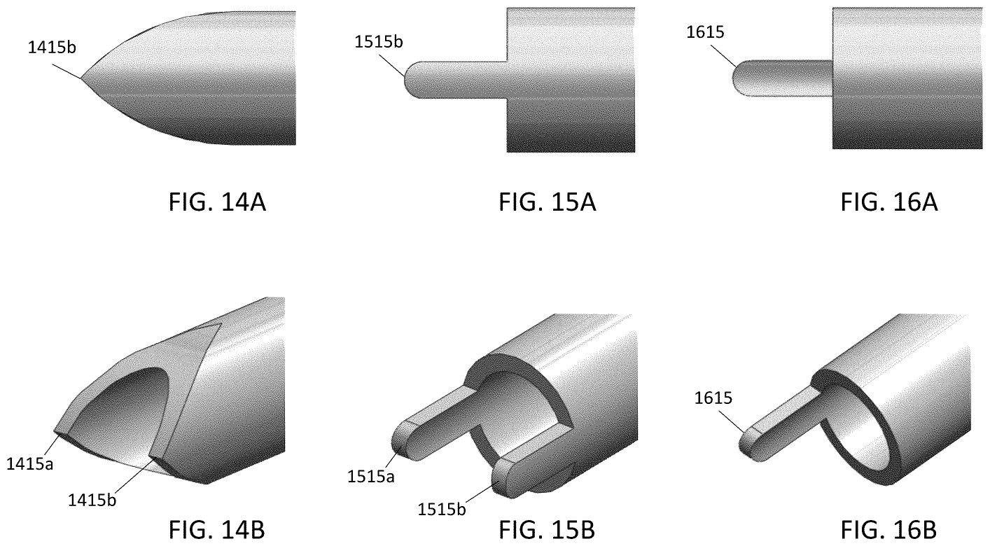

FIGS. 14A and 14B show an example distal end of a first elongate member having two protruding portions 1415a and 1415b.

FIGS. 15A and 15B show an example distal end of a first elongate member having two protruding portions 1515a and 1515b with rounded ends.

FIGS. 16A and 16B show an example distal end of a first elongate member having a protruding portion 1615 with a rounded end.

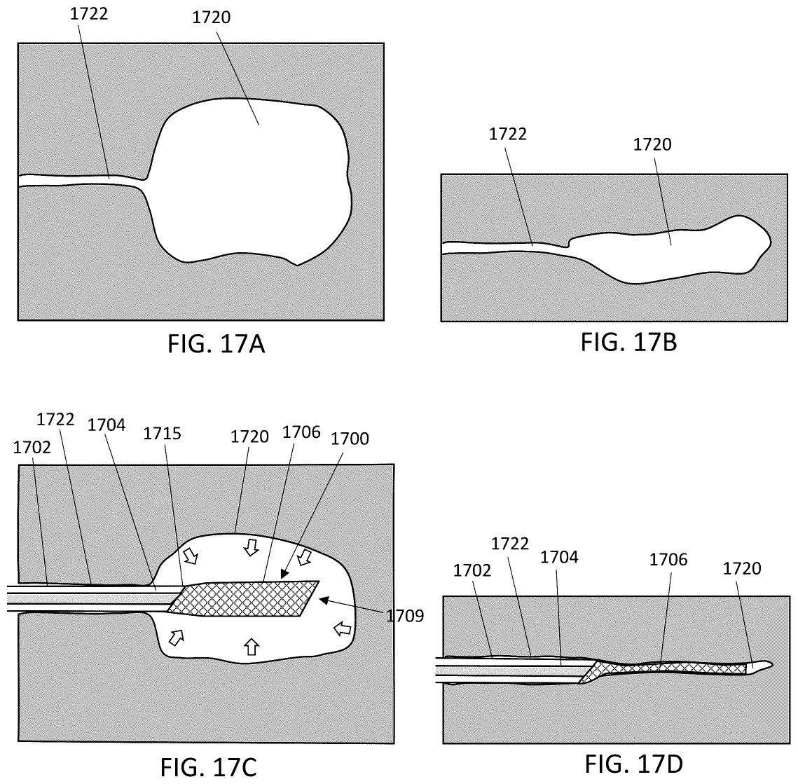

FIGS. 17A-17D show an example use of an example surgical drain in a soft tissue region of a body. FIG. 17A shows a first cross sectional view of the soft tissue region, which includes a cavity 1720 and a channel 1722 that leads to the cavity 1720. The soft tissue region may be a surgical site, such as a postpartum uterus or a site of removal for tumor. For example, the channel 1722 may include a portion of a vaginal canal and the cavity 1720 may include a postpartum uterus. FIG. 17B shows a second sectional view (e.g., taken at 90 degrees offset from the view shown in FIG. 17A). As shown, tissue may be open more in one direction than another.

FIG. 17C shows the surgical drain 1700 after being inserted within the body region and as a negative pressure (suction) is applied. As shown, the mesh 1706 is positioned within the cavity 1720 and a first elongate member 1702 is positioned within the channel 1722. In the example shown, the mesh 1706 and a second elongate member 1704 are advanced through a lumen of the first elongate member 1702. For example, the first elongate member 1702, with the mesh 1706 positioned therein, may be advanced within the channel 1722, then the mesh 1706 may be distally extended out of the first elongate member 1702 and into the cavity 1720 by pushing the second elongate member 1704 relative to the first elongate member 1702. In other examples, the second elongate member 1704 is not used and the mesh 1706 may be pushed directly through the first elongate member 1702.

The distal end 1709 of the first elongate member 1702 is tapered to define a distally protruding portion 1715 that is arranged to asymmetrically distribute a compressive stress placed on the mesh 1706 and to prevent the mesh 1706 from binding or jamming as the mesh 1706 exits the distal opening of the first elongate member 1702. In some cases, the mesh 1706 may be manipulated to take on a double-walled tubular configuration when extended into the cavity 1720, such as shown in the examples of FIGS. 3B, 4B, 5B and 7B.

The first elongate member 1702 may form a seal with the walls of the soft tissue of the channel 1722 so that sufficient negative pressure can form within the cavity 1720. In some cases, the first elongate member 1702 includes one or more sealing features (e.g., seals and/or plugs) to facilitate the sealing, as described herein.

The first elongate member 1702 may be any appropriate length so that it may be manipulated and position the mesh 1706 within the body region being treated. For example, the first elongate member 1702 may be between 5 cm and 100 cm long (e.g., between 10 cm and 50 cm, between 10 cm and 35 cm, etc.). The first elongate member 1702 may be straight (as shown) or curved, including curved with a fixed curve (e.g., between 10-80 degrees). In some cases, the first elongate member 1702 and/or second elongate member 1704 may be laterally flexible.

In some cases, the second elongate member 1704 extends distally at least partially within the mesh 1706. In other cases, the second elongate member 1704 does not extend distally within the mesh 1706. In some examples, the second elongate member 1704 may be concentrically arranged within the first elongate member 1702, and may be coupled to one end of the mesh 1706. The mesh 1706 may be radially compressible such that the outer diameter of the mesh 1706 is sufficiently reduced for entry into the soft tissue region. The mesh 1706 may be flexible and laterally deflectable (i.e., bendable) to conform to the anatomy of the body tissue. As described herein, the mesh 1706 may include a plurality and/or network of pores (e.g., open cell structure) that is configured to draw fluid, air and/or solid materials from a body cavity.

In some examples the mesh 1706 may be compressed into a compressed state within the first elongate member 1702. Once released from the first elongate member 1702 and extended within the cavity 1720, the mesh 1706 may expand into an expanded state. In some cases, the mesh 1706 may at least partially change shape (e.g., bend) when inserted within the cavity 1720, for example, by pressure from contact with surrounding tissue. In some cases, the mesh 1706 may be configured to take on a pre-determined shape (e.g., bent shape), for example, to conform to a shape of a particular body cavity.

In some examples, the first elongate member 1702 and/or the second elongate member 1704 may include one or more stops that limit their relative axial movement. For example, the first elongate member 1702 and the second elongate member 1704 may be configured to lock with respect to each other when the mesh 1706 extends distally and/or retracts proximally by a predetermined amount. In some examples, the drain includes one or more locks configured to releasably lock the relative axial positions of the first elongate member 1702 and the second elongate member 1704.

Once the mesh 1706 is deployed within the cavity 1720, a negative pressure may be applied through the lumen of the first elongate member 1702 to cause fluid, gas and/or other bodily material from the cavity 1720 to flow proximally through the mesh 1706, into the first elongate member 1702, and eventually out of the body tissue. For example, the first elongate member 1702 may include one or more openings at the distal end of the first elongate member (and/or within a side wall in a distal region of the first elongate member 1702). Typically the suction lumen through the first elongate member 1702 may be in fluid communication with the mesh 1706 (e.g., with the second lumen of formed by a double-wall tubular configuration). The mesh 1706 can maintain a shape that provides efficient flow of fluid, gas and/or other bodily material through the network of pores of the mesh 1706, even when compressed by the tissue, as shown in FIG. 17D. The negative pressured applied by the mesh 1706 may apply an inward force on the surrounding walls of the cavity 1720 (indicated by inward facing arrows in FIG. 17C), thereby causing the cavity 1720 (e.g., uterus) to at least partially contract. Such contraction may be beneficial, for example, in cases where contracting a postpartum uterus may reduce hemorrhaging.

In some cases where the mesh 1706 has tubular shape, application of the negative pressure may flatten the outer shape of the tube, creating a flattened tube shape. However, the pores of the mesh 1706 may sufficiently maintain their shape to allow fluid, material and/or air to pass therethrough.

The negative pressure may be maintained for a period of time to provide a therapeutic benefit. For example, the negative pressure may be applied until the cavity 1720 is sufficiently drained of fluid and/or the cavity 1720 is sufficiently contracted. In some examples, the period of time may range from one minute to several hours or even days. For example, the period of time may range from one minute to 5 days or more (e.g., 10 minutes, 30 minutes, 1 hour, 2 hours, 3 hours, 4 hours, 5 hours, 6 hours, 8 hours, or 10 hours, 12 hours, 18 hours, 24 hours, 48 hours, 3 days, 4 days, 5 days, etc.).

The mesh 1706 may be removed from the cavity 1720 by proximally moving the mesh 1706 out of the cavity 1720. For example, the second elongate member 1704 may be pulled proximally to retract the mesh 1706 within the first elongate member 1702. Retraction into the first elongate member 1702 may cause the mesh 1706 to radially contract thereby exerting a compressive stress on the mesh 1706. In other examples where the second elongate member 1704 is not used, the first elongate member 1702 may be pulled to directly pull the mesh 1706 out of the cavity 1720. The distally protruding portion 1715 of the first elongate member 1702 is arranged to asymmetrically distribute a compressive stress placed on the mesh 1706 and to prevent the mesh 1706 from binding or jamming as the mesh 1706 enters the distal opening of the first elongate member 1702.

In some examples, the negative pressure is optionally maintained within the cavity 1720 for a period of time after withdrawing the mesh 1706 from the cavity 1720. For example, in some cases, maintaining the negative pressure after removal of the mesh 1706 may help to contract a uterus and mitigate uterine hemorrhaging. In some examples, the negative pressure may be applied period of time may range from one minute to 10 hours (e.g., 10 minutes, 30 minutes, 1 hour, 2 hours, 3 hours, 4 hours, 5 hours, 6 hours, 8 hours, or 10 hours) after withdrawal of the mesh 1706 from the cavity 1720. The negative pressure be applied via the first elongate member 1702 after withdrawing the mesh 1706. Once the treatment is complete, the mesh 1706 and first elongate member 1702 may be removed proximally from the cavity 1720 and the channel 1722.

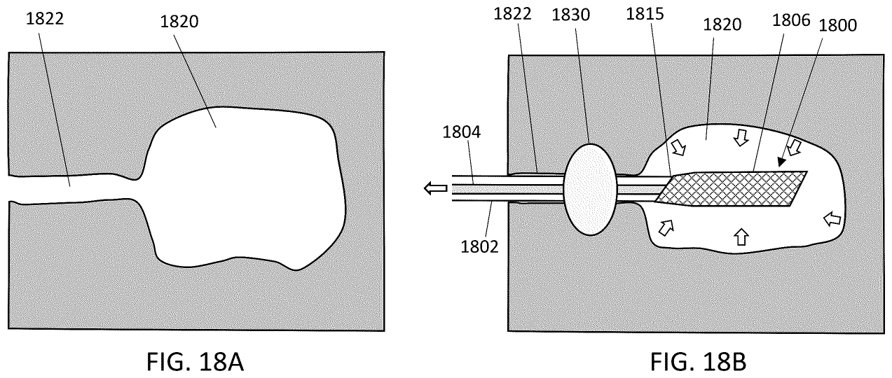

As described herein, in some examples the surgical drain includes one or more plugs to assist in providing a seal to provide a negative pressure to a body cavity. FIGS. 18A-18B show an example use of an example surgical drain 1800 having a plug 1830 for sealing with soft tissue. FIG. 18A shows a cross section view of a soft tissue region of a body that includes a cavity 1820 and a channel 1822 that leads to the cavity 1820. The soft tissue region may be a surgical site, such as a postpartum uterus or a site of removal for tumor. For example, the channel 1822 may include a portion of a vaginal canal and the cavity may include a postpartum uterus. In this example, the channel 1822 has a larger diameter than the channel 1722 in FIGS. 17A-17D.

FIG. 18B shows the surgical drain 1800 after being inserted within the body region and as a negative pressure is applied. The surgical drain 1800 includes a first (e.g., outer) elongate member 1802 and a second (e.g., inner) elongate member 1804 and an invertible tubular mesh 1806 coupled to a distal region of the second elongate member 1804. In addition, a plug 1830 (also referred to as an occluder) is positioned around a portion of the first elongate member 1802 proximal to the mesh 1806. The plug 1830 may be made of an elastic body made of an clastic material (e.g., foam or sponge) that can be radially compressed for positioning within the channel 1822 and that can expand within the channel 1822 to create a seal against tissue walls. In some cases, the plug 1830 may include one or more outer covers or sheaths that is/are arranged to apply a radial compressive force on the elastic body to reduce an outer diameter of the plug 1830, for example, for positioning the plug 1830 within the channel 1822. As described herein, the outer cover(s) of the plug 1822 may be slidably coupled to the first elongate member 1802 to control application of the radial compressive force on the elastic body.

The plug 1830 may be arranged such that the lumen of the first elongate member 1802 passes through the plug 1830, thereby allowing the second elongate member 1804 to pass through the plug 1830 and for suction to be applied to the mesh 1806 via the first elongate member 1802 (and/or the second elongate member 1804).

The first elongate member 1802 with the plug 1830 is positioned within the channel 1822. The diameter of the first elongate member 1802 may not be large enough to seal off the cavity 1820. However, the plug 1830 can be expanded within the channel 1822 to form a seal with the walls of the soft tissue walls of the channel 1822 so that sufficient negative pressure can form within the cavity 1820. The mesh 1806 is extended from the first elongate member 1802 so that it can expand into the cavity 1820. As described herein, the distal end of the first elongate member 1802 includes a distally protruding portion 1815, which may prevent an inversion region of the mesh 1806 from binding or jamming as it is pushed through the distal opening of the first elongate member 1802. Once the mesh 1806 is extended and released within the cavity 1820, a negative pressure may be applied (e.g., through a lumen of the first elongate member 1802 and/or the second elongate member 1804) to cause fluid, gas and/or other material from the cavity 1820 to flow proximally through the mesh 1806 and eventually out of the body tissue. Alternatively or additionally, the negative pressure applied by the mesh 1806 may apply an inward force on the surrounding walls of the cavity 1820 (indicated by inward facing arrows), thereby causing the cavity 1820 (e.g., uterus) to at least partially contract (e.g., as shown in FIG. 17D).

After a sufficient time of negative pressure, the mesh 1806 may be retracted from the cavity 1820. For example, the second elongate member 1804 may be pulled relative to the first elongate member 1802 to pull the mesh 1806 within the lumen of the first elongate member 1802. The distally protruding portion 1815 may prevent the inversion region of the mesh 1806 from binding or jamming as it is pulled through the distal opening of the first elongate member 1802. In some examples, the negative pressure is optionally maintained within the cavity 1820 for a period of time after withdrawing the mesh 1806 from the cavity 1820. Once the treatment is complete, the mesh 1806, the second elongate member 1804 and the first elongate member 1802 may be removed proximally from the cavity 1820 and the channel 1822. The plug 1830 may be radially compressed prior to removal of the first elongate member 1802.

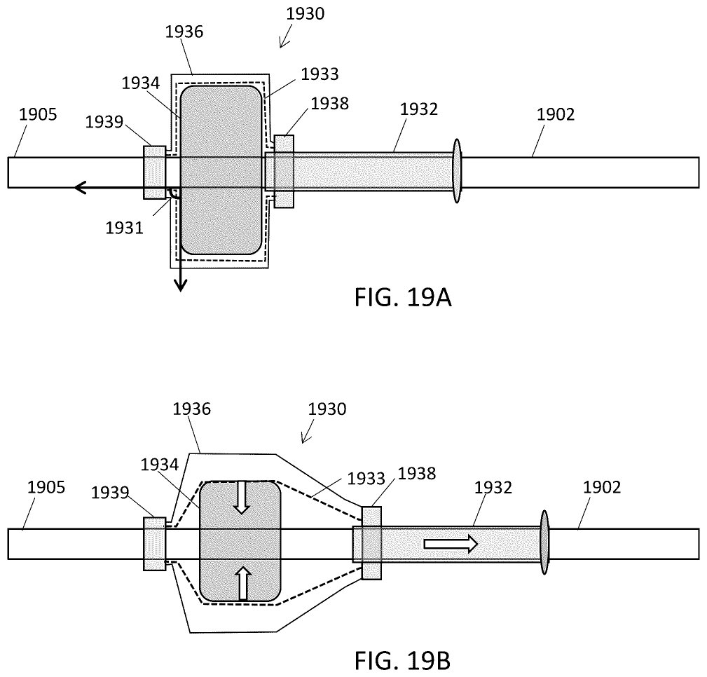

FIGS. 19A and 19B show an example plug assembly 1930. In this example, an outer surface of an elastic body 1934 is covered with a covering, which includes a compression layer 1933 and a fluid barrier layer 1936. FIG. 19A shows the plug 1930 in a radially expanded state and FIG. 19B shows the plug 1930 in a radially compressed state. The plug 1930 is positioned around a first elongate member 1902. Distal ends of the compression layer 1933 and the fluid barrier layer 1936 are fixedly coupled to the first elongate member 1902 via a first (e.g., distal) connecter 1939. Proximal ends of the compression layer 1933 and the fluid barrier layer 1936 are slidably coupled to the first elongate member 1902 to a second (e.g., proximal) connector 1938. In this example, the second connector 1938 has an elongated proximal side 1932 that may serve as a handle. The first connecter 1939 and the second connector 1938 may be referred to as cuffs or collars. In some examples, the first connecter 1939 and the second a connecter 1938 include bands (e.g., clastic bands) and/or washers. Driving the second connector 1938 in a proximal direction (e.g., by pulling the handle 1932 by hand or by an actuator), as shown in FIG. 19B, causes the compression layer 1933 to elongate and apply a radial compression force on the clastic body 1934, thereby reducing the outer diameter of the elastic body 1934 and reducing a diameter of the plug 1930. Releasing the proximal axial force placed on the second connector 1938 (e.g., by releasing the handle 1932) creates slack the compression layer 1933 and releases the radial compression force, thereby reducing the outer diameter of the elastic body 1934 and the plug 1930 as shown in FIG. 19B. In some examples, the radially expanded state of the elastic body 1934 (FIG. 19A) may be reinforced based on an extent to which the second connector 1938 is axially displaced distally and an amount of axial force placed on the elastic body 1934 distally. For example, a sufficient distal axial force may be placed on second connector 1938 to axially compress the elastic body 1934, which may stiffen and reinforce the elastic body 1934 in the expanded state.

In some cases, the plug assembly 1930 may include one or more stops and/or locks that limit and/or lock the axial position of the second a connecter 1938 (and the handle 1932) relative to the first connecter 1939. For example, the second connecter 1938 may include a lock that releasably locks an axial position of the second connecter 1938 relative to the elongate member 1902. Thus, in the case of lock(s), the clastic body 1934 may be locked in an a radially expanded state (e.g., FIG. 19A) or a radially compressed state (e.g., FIG. 19B).

The fluid barrier layer 1936 may be a thin layer of fluid resistant material. In some examples, the fluid barrier layer 1936 may be made of polyethylene (e.g., light weight polyethylene), nitrile, or nitrile-like low stretch material. The thickness of the fluid barrier layer 1936 may vary depending on the material. In some examples, the fluid barrier layer 1936 has a thickness ranging between about 0.0001 inches to 0.01 inches). The compression layer 1933 may have a relatively high tensile strength so that it can apply a radially inward pressure on the elastic body 1934. In some cases, the compression layer 1933 is a tubular mesh. In one example, the compression layer 1933 is a tubular fabric braid having a diameter ranging between about 3 inches and 5 inches and with about 100-200 monofilaments having diameters ranging between about 0.005 inches and 0.1 inches. The elastic body 1934 may be made of any of a number of elastic materials, such as a polymer foam or sponge material. In some examples, the clastic body 1934 has flat sides (in the axial direction) to create predetermined angle 1931 between the elastic body 1934 and the first elongate member 1902. In some cases, the predetermined angle is about 90 degrees (perpendicular), which may allow for the most diameter change of the elastic body 1934 per pull length.

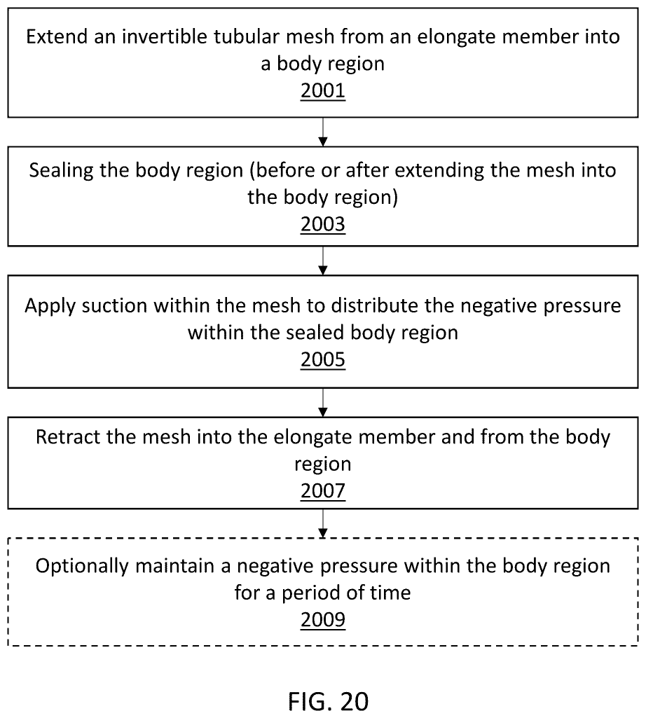

FIG. 20 is a flowchart indicating an example method of treating a body region using a surgical drain as described herein. The body region may be a wound, body cavity, a canal, a channel, or post-partum uterus. The method includes extending an invertible tubular mesh from an elongate member (e.g., first elongate member) and into the body region 2001. The mesh may be configured to fold and invert at an inversion region of the mesh. A distal end of the elongate member may be shaped to prevent binding or jamming of the inversion region of the mesh as the mesh is extended out of a distal opening of the elongate member, as described herein. The mesh may have pores of sufficient size to allow passage of fluids (e.g., blood, lymph, pus), gasses and/or other materials (e.g., coagulate, etc.) to pass without significant resistance. The mesh may be configured to expand when released from the elongate member and take on a shape that distributes a negative pressure within the body region. In some cases, the mesh is adjusted to take on a double-walled tubular configuration where the porous wall doubles back on itself to form a second lumen, as described herein.

Before, during or after releasing the mesh within the body region, the method includes sealing the body region 2003. The surgical drain may include one or more sealing features to create a seal sufficient to hold suction. In some examples, the elongate member (e.g., first elongate member) includes a plug proximally positioned with respect to the distal porous drain that has an expandable outer diameter to create a seal with surrounding tissue (e.g., within a body channel). The plug may include an inner elastic, e.g., viscoelastic foam, body and a compression layer surrounding the elastic body and configured to apply a compression force to reduce the diameter of the elastic body. The elastic body may be configured to radially compress and/or fold to reduce the diameter of the plug (e.g., for insertion within the channel). The elastic body may be made of an elastic material, such as a foam (e.g., porous polymer material). The plug may optionally include a fluid barrier (e.g., layer) to prevent fluid from contacting the clastic body and/or compression layer. The plug may optionally include a lock that is configured to lock the plug in a radially expanded and/or compressed state. The apparatus may be configured to activate the plug by a handle that is configured to elongate, shorten and/or twist the compression layer.

Once the body region is sealed, suction may be applied through the mesh 1905. For example, the elongate member may be operationally coupled to a vacuum source for applying a vacuum through the lumen of the elongate member and the mesh. In some cases, the suction may be maintained for between about 1 minute and 5 days or more (e.g., 1 minute, 5 minutes, 10 minutes, 1 hour, 2 hours, 3 hours, 4 hours, 5 hours, 6 hours, 12 hours, 24 hours, 2 days, 3 days, 4 days, etc.).

Once the body region is drained and/or contracted, and sufficient suction has been applied, the mesh may be retracted into the elongate member. The mesh may be pulled proximally, for example, by pulling a second (inner) elongate member coupled to the mesh. The tubular mesh may invert at an inversion region of the mesh as it is withdrawn back into the distal opening of the elongate member. The shaped distal end of the elongate member may prevent binding or jamming of the inversion region as the mesh is radially compressed and retracted into the distal opening of the elongate member, as described herein. In some examples, the suction is optionally maintained within the body region via the elongate member for a period of time 2009 after withdrawing the mesh from the body region. In certain situations this may help to contract the body region and mitigate hemorrhaging, such as uterine hemorrhaging in some postpartum situations.

Any of the meshes described herein may have an open pore structure in which pores/holes/spaces within a mesh are interconnected to provide multiple channels throughout the mesh. In some examples, the mesh includes a porous material (e.g., fabric and/or textile), which may include woven, knitted or braided elements (e.g., filaments). In some examples, the mesh may be formed of a knit, a weave, a braid, a non-woven sheet (e.g., polymer or metallic or mixes), or a flexible tube of material having pores. For example, in variations in which the mesh is formed of a braided material, the braid may include any number of filaments, e.g., between 24-144 ends/filaments (e.g., between about 24-128 filaments, between about 32-98 filaments, etc.). In some examples, the filaments are formed of a material such as PET, Nylon, PP, Nitinol, Steel, Elgiloy, or some combination of these. The filament may be any appropriate diameters, such as between 0.003″ to 0.025″ diameter filaments (e.g., monofilaments or compound filaments). In some examples, the mesh is formed of filaments (knit, woven, braided, etc.) of between 100-2000 denier (e.g. multifilament or monofilament). The mesh may have a mono or multi filament structure (or a mixture thereof).

In some examples, the mesh is made of a non-woven material, such as a punched material, slitted material, felt, melt blown material and/or foam material. For example, the mesh may be formed by extrusion, punching, stamping, blowing, laser cutting and/or other manufacturing techniques. In some examples, the mesh may include an open cell structure (e.g., open cell or reticulated foam) that includes interconnected holes/spaces (e.g., cells). In some cases, the foam is similar to some types wound dressing foams used with negative pressure. In some cases, the foam may be reinforced with open textile structure (e.g., net-like tubes, sheets) to hold the foam together when placed under tension. For example, the foam may be a composite foam, or a fabric covered foam. In some examples, the mesh includes a pore pattern, for example, with 1 mm to 4 mm holes (e.g., like as perforated structures with many holes per unit area). In some examples, the mesh includes a pattern of slits, for example, with slits with 1 mm width to 3 mm width by 1 mm length to 15 mm length.

The meshes described herein may be made of any of a number of biocompatible materials. In some examples, the mesh includes one or more polymers, such as polyethylene terephthalate (PET), polytetrafluoroethylene (PTFE), silicone and/or polyurethane. In some cases, the PTFE is an expanded polytetrafluoroethylene (ePTFE). In some cases, the polymer includes a thermoplastic or thermoset material (e.g., thermoplastic or thermoset foam). In some examples, the mesh includes one or more metals (e.g., metal filaments), such as nickel titanium alloy (e.g., nitinol), steel, Elgiloy and/or nickel-cobalt-chromium-molybdenum alloy (e.g., MP35N).

The term “mesh” is not limited to structures formed by one or more strands, but may be formed of a non-woven material. The material forming the mesh may be a porous filtering material such as Tyvek, filter paper, etc. or it may be (initially) non-porous and pores may be formed therein. The term “mesh” may refer to a material having an average porosity of greater than 50% that may be formed into an inverting structure that is sufficiently compliant so that it may invert back over itself. The mesh may be formed as a tubular or basket shape (e.g., open at both ends or closed at one end (e.g., the distal end). In some cases, the mesh may be shaped into a generally tubular shape (open at one or both ends). In some cases, the mesh may be shaped into a non-tubular shape.

In any of the apparatuses described herein, the mesh may be made of a flexible porous material. In some examples, the mesh may be a fabric. The mesh may be formed of filaments (e.g., strands) of material, such as monofilaments or multiple filaments. For example, the mesh may comprise a braided polymeric monofilament having 24 or more strands (e.g., 30 or more strands, 34 or more strands, 36 or more strands, 38 or more strands, 40 or more strands, 42 or more strands, etc.).

The mesh typically has a plurality of openings or pores where the pores are sufficiently large to allow fluids and some solid biological debris (e.g., clots, pus, coagulate) to pass easily. For example, the pores may have a pore diameter that is 0.1 mm or greater (0.2 mm or greater, 0.3 mm or greater, 0.4 mm or greater, 0.5 mm or greater, 0.6 mm or greater, 0.7 mm or greater, 0.8 mm or greater, 0.9 mm or greater 1 mm or greater, 1.1 mm or greater, 1.2 mm or greater, 1.3 mm or greater, 1.4 m or greater, etc.). The pores may be formed by the spaces between the strands, e.g., in woven, braided and/or knitted mesh.

The meshes described herein may have any of a number of shapes. In some examples, the mesh may have a tubular shape with an inner space (e.g., lumen). In some cases, the mesh may include multiple tubes of porous material (e.g., concentrically arranged).

In any of the apparatuses, the first elongate member (e.g., tube or catheter) and/or the second elongate member (e.g., rod or inner tube or catheter), may be flexible, semi-ridged or rigid. For example, the elongate member(s) may be formed of polyurethane or silicone. The surgical drains may be configured to have reasonably high column force while retaining bending flexibility. For example, they may have sufficient axial flexibility so that they can be bent around structures or non-uniform volumes within soft tissue.

In any of the examples, the proximal direction may generally be in the direction towards the hand of the user (e.g., physician, surgeon, medical technician, nurse, etc.) operating the device, and distal may generally be in the direction away from the hand of the user.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A surgical drain system, comprising:
a first elongate member having a distal end defining a distal opening that provides access to a lumen of the first elongate member, wherein the distal end includes a distally protruding portion;
a second elongate member that is slidably disposed in the lumen; and
a tubular mesh having a first end coupled to the first elongate member and a second end coupled to the second elongate member, wherein the mesh is configured to transition between an expanded configuration, in which the mesh is expanded distally past the distal end of the first elongate member, and a retracted configuration, in which the mesh is inverted and withdrawn within the lumen of the first elongate member, wherein the mesh is configured to invert at an inversion region as the mesh is extended from or retracted into the distal opening of the first elongate member, and
wherein the distally protruding portion of the first elongate member is arranged to axially distribute stress along the inversion region of the mesh as the mesh is extended from or retracted into the distal opening, thereby reducing binding of the mesh.

2. The surgical drain system of claim 1, wherein the distal end of the first elongate member has a tapered shape that defines the distally protruding portion.

3. The surgical drain system of claim 2, wherein the tapered shape has an angle ranging between 10 degrees and 45 degrees with respect to a longitudinal axis of the first elongate member.

4. The surgical drain system of claim 2, wherein the tapered shape has an angle ranging between 30 degrees and 45 degrees with respect to a longitudinal axis of the first elongate member.

5. The surgical drain system of claim 2, wherein the tapered shape has an angle ranging between 1 degree and 89 degrees with respect to a longitudinal axis of the first elongate member.

6. The surgical drain system of claim 1, wherein the distally protruding portion of the first elongate member is arranged to asymmetrically distribute the stress along an inversion plane of the inversion region of the mesh.

7. The surgical drain system of claim 1, wherein the distally protruding portion of the first elongate member is arranged to provide an axially elongated distal opening of the first elongate member.

8. The surgical drain system of claim 1, wherein the distal end of the first elongate member includes multiple distally protruding portions arranged to asymmetrically distribute stress along the inversion region of the mesh as the mesh is extended from or retracted into the distal opening.

9. The surgical drain system of claim 1, further comprising a backstop configured to limit an extent to which the second elongate member is allowed to be pulled proximally.

10. The surgical drain system of claim 1, wherein an outer surface of the first elongate member comprises a plug that is configured to compress and expand, wherein when expanded, the plug is shaped to create a seal with surrounding soft tissue.

11. The surgical drain system of claim 1, wherein the first end of the mesh is coupled to a distal end region of the first elongate member, and wherein the second end of the mesh is coupled to a distal end region of the second elongate member.

12. The surgical drain system of claim 11, wherein the mesh is coupled to an outer surface of the distal end region of the first elongate member.

13. The surgical drain system of claim 1, wherein the mesh is configured to transition from the expanded configuration to the retracted configuration upon application of a pulling force on the second elongate member.

14. The surgical drain system of claim 1, wherein the mesh is configured to transition from the retracted configuration to the expanded configuration upon application of a pushing force on the second elongate member.

15. The surgical drain system of claim 1, wherein the first elongate member is a tube.

16. The surgical drain system of claim 1, wherein the second elongate member is a tube or a rod.

17. The surgical drain system of claim 1, wherein the lumen of the first elongate member is configured to act as a vacuum channel configured to create a suction for removing fluid from a soft tissue cavity when the mesh is positioned within the soft tissue cavity.

18. The surgical drain system of claim 17, wherein the vacuum channel extends between the distal end of the first elongate member and one or more proximal vacuum ports of the first elongate member.

19. The surgical drain system of claim 17, wherein the vacuum channel is within a space between an outer surface of the second elongate member and an inner surface of the first elongate member.

20. The surgical drain system of claim 1, wherein a lumen of the second elongate member is configured to act as a vacuum channel configured to create a suction for removing fluid from a soft tissue cavity when the mesh is positioned within the soft tissue cavity.

21. A method of draining a body region, the method comprising:
positioning a surgical drain apparatus into a soft tissue channel that leads to the body region, wherein the surgical drain apparatus includes a first elongate member having a distal end defining a distal opening that provides access to a lumen of the first elongate member, wherein the distal end includes a distally protruding portion, and wherein a tubular mesh is housed within the lumen of the first elongate member;
distally extending the tubular mesh from the lumen of the first elongate member and into the body region, wherein distally extending the tubular mesh causes the tubular mesh to transition from an inverted configuration to a non-inverted configuration, wherein the tubular mesh inverts at an inversion region as the mesh is extended from distal opening of the first elongate member, and wherein the distally protruding portion of the first elongate member axially distributes stress along the inversion region of the mesh as the mesh is extended from the distal opening, thereby preventing binding of the mesh; and
applying suction through the lumen so that a plurality of flow paths are created through the mesh, wherein the suction drains material proximally from the body region through the mesh and the first elongate member.

22. The method of claim 21, further comprising, after applying sufficient suction to the body region, retracting the tubular mesh into the first elongate member, wherein the distally protruding portion of the first elongate member asymmetrically distributes the stress along the inversion region of the mesh as the mesh is retracted into the distal opening, thereby preventing binding of the mesh.

23. The method of claim 21, further comprising, prior to applying the suction, creating a seal around the first elongate member with the soft tissue channel to maintain a vacuum within the body region.

24. The method of claim 21, wherein the surgical drain apparatus further comprises a second elongate member slidably disposed within the lumen of the first elongate member and coupled to the tubular mesh, wherein distally extending the tubular mesh from the lumen of the first elongate member comprises pushing the second elongate member distally.

25. The method of claim 21, wherein the distal end of the first elongate member has a tapered shape that defines the distally protruding portion.

26. The method of claim 25, wherein the tapered shape has an angle ranging between 10 degrees and 45 degrees with respect to a longitudinal axis of the first elongate member.

27. The method of claim 25, wherein the tapered shape has an angle ranging between 30 degrees and 45 degrees with respect to a longitudinal axis of the first elongate member.

28. The method of claim 25, wherein the tapered shape has an angle ranging between 1 degree and 89 degrees with respect to a longitudinal axis of the first elongate member.

29. The method of claim 21, wherein the distally protruding portion of the first elongate member asymmetrically distributes the stress along an inversion plane of the inversion region of the tubular mesh.

30. The method of claim 21, wherein the distally protruding portion of the first elongate member axially elongates the distal opening of the first elongate member.

31. The method of claim 21, wherein the distal end of the first elongate member includes multiple distally protruding portions that asymmetrically distribute the stress along the inversion region of the mesh as the mesh is extended from the distal opening.
